(19) United States
(12) Patent Application Publication
(10) Pub. No.: US 2026/0196316 A1
(43) Pub. Date: Jul. 9, 2026

(54) PATIENT INFORMATION RECORDING METHOD AND SYSTEM

(71) Applicant: Hunan Provincial Hospital of Integrated Traditional Chinese and Western Medicine, Changsha (CN)

(72) Inventors: Yunfeng Duan, Changsha (CN); Haili Zhu, Changsha (CN); Juhua Hu, Changsha (CN)

(21) Appl. No.: 19/072,052

(22) Filed: Mar. 6, 2025

(30) Foreign Application Priority Data
May 14, 2024 (CN) 2024105930674

(51) Int. Cl. G16H 10/65; A41D 13/12; A41D 27/20

(52) U.S. Cl. CPC G16H 10/65; A41D 13/1209; A41D 27/205

(57) ABSTRACT

A patient information recording method and system are provided, which relate to the field of data processing technologies. The method includes: obtaining patient medical record information; constructing an entity recognition model based on a named entity recognition algorithm and a conditional random field algorithm; performing semantic extraction on the patient medical record information through the entity recognition model to obtain to-be-recorded patient information; classifying the to-be-recorded patient information to generate a category of the to-be-recorded patient information; generating an input window for patient identity information based on an XSL file; where the XSL file includes an XSLT tool and an XPath tool, and the input window includes multiple operation buttons for the to-be-recorded patient information; receiving the patient identity information; displaying multiple input sub-windows associated with the patient identity information; receiving user input through input sub-windows; and displaying the user input according to the category of the to-be-recorded patient information.

### CROSS-REFERENCE TO RELATED APPLICATION

This application claims priority to Chinese Patent Application No. 202410593067.4, filed on May 14, 2024, which is herein incorporated by reference in its entirety.

### TECHNICAL FIELD

The disclosure relates to the field of data processing technologies, and more particularly to a patient information recording method and a patient information recording system.

### BACKGROUND

Patient information recording refers to a process of medical staff recording patient-related information during work. This information includes personal information, medical history, condition changes, treatment plan and medication status of the patient. A purpose of recording this information is to provide patients with comprehensive medical services, continuously monitor health status of the patient, evaluate treatment effect, coordinate work of the medical team, and ensure patient safety.

The importance of patient information recording lies in providing a basis for medical decision-making and helping medical staff to better manage and treat patients. With the development of medical technology, information tools such as electronic medical record systems have gradually replaced traditional paper records, which improves efficiency and accuracy of information recording. At the same time, it is also easier for the medical staff to access and share patient information, which improves the quality and efficiency of medical services.

At present, the medical staff generally records relevant patient information through paper notebooks or general electronic devices, which are not very targeted. When recording the patient information, the medical staff need to hold the electronic devices and need to remember a large amount of the patient information that needs to be recorded. It is inconvenient for the medical staff to use and has a heavy memory burden. In addition, the recorded patient data often has a large difference in order and is difficult to access.

### SUMMARY

In order to solve technical problems in the related art that medical staff generally records relevant patient information through paper notebooks or general electronic devices, which are not very targeted, when recording the patient information, the medical staff need to hold the electronic devices and need to remember a large amount of the patient information that needs to be recorded, which is inconvenient for the medical staff to use and has a heavy memory burden, and the recorded patient data often has a large difference in order and is difficult to access, the disclosure provides a patient information recording method and system.

Technical solutions provided by embodiments of the disclosure are as follows.

In the first aspect, the embodiments of the disclosure provide a patient information recording method, applied to a portable electronic recording device. The portable electronic recording device is detachably disposed on a sleeve of working clothes through a single-sided transparent bag, and the portable electronic recording device is connected to a hospital information system (HIS). The patient information recording method includes:
S1, obtaining patient medical record information;
S2, constructing an entity recognition model based on a named entity recognition (NER) algorithm and a conditional random field (CRF) algorithm, where the entity recognition model includes an input module, a first module based on the NER algorithm, a second module based on the CRF algorithm and an output module sequentially connected in that order;
S3, performing semantic extraction on the patient medical record information through the entity recognition model to obtain patient information to be recorded, where the patient information to be recorded includes patient identity information;
S4, classifying the patient information to be recorded to generate a category of the patient information to be recorded;
S5, generating an input window related to the patient identity information based on an extensible stylesheet language (XSL) file, where the input window includes input sub-windows bound to the patient information to be recorded, the XSL file includes an extensible stylesheet language transformation (XSLT) tool and an XML path language (XPath) tool, and the input window includes multiple operation buttons related to the patient information to be recorded;
S6, receiving the patient identity information;
S7, displaying multiple input sub-windows associated with the patient identity information;
S8, receiving user input through the multiple input sub-windows; and
S9, displaying the user input according to the category of the patient information to be recorded.

In the second aspect, the embodiments of the disclosure provide a patient information recording system, including a processor and a memory. The memory is stored with computer-readable instructions, and the computer-readable instructions are configured, when being executed by the processor, to implement the patient information recording method as described in the first aspect.

In the third aspect, the embodiments of the disclosure provide a non-transitory computer-readable medium stored with a computer program, the computer program is configured, when being executed by a processor, to implement the patient information recording method as described in the first aspect.

The beneficial effects brought by the technical solutions provided by the embodiments of the disclosure at least include the follows.

In the disclosure, the entity recognition model is constructed based on the NER algorithm and the CRF algorithm. Specifically, the patient information to be recorded is recognized through the NER algorithm, then the recognized patient information to be recorded is checked with correct patient information to be recorded through the CRF algorithm, and the final patient information to be recorded is determined through multiple screenings, so as to reduce deviation between the patient medical record information and the recording information, and ensure the accuracy of the patient information to be recorded obtained according to the patient medical record information. This method also can be applied to electronic medical record information in different formats from different hospitals, can automatically extract the patient information to be recorded, and reduce the memory burden of the medical staff when collecting the patient information. The obtained patient information to be recorded is classified for the second time, so that the similar patient information to be recorded is classified into the same larger category, which is convenient for the medical staff to collect and access the patient information, and avoids confusion in information management. Finally, the input sub-windows bound to the patient information to be recorded are generated by using the XSL file. The input window generated by the XSL file can be easily customized according to the needs to make the interface simple and beautiful. Such input interface can make the medical staff more focused on recording the patient information, and reduce unnecessary visual interference. The patient information to be recorded is bound to the input sub-windows, and the operation buttons are provided at the same time, so that the medical staff can record the patient information intuitively and conveniently, thereby improving the work efficiency and recording quality, avoiding medical problems caused by deviations in manual memory of the medical staff, and improving information management efficiency and accuracy. In addition, the portable electronic recording device is detachably disposed on the sleeve of the work cloth through the single-sided transparent bag, without the need to hold a notebook or recorder. The portable electronic recording device is connected to the HIS system, which can greatly release the work efficiency of the medical staff and ensure the integrity of the recorded patient information.

### BRIEF DESCRIPTION OF DRAWINGS

In order to describe technical solutions in embodiments of the disclosure clearer, drawings required in the descriptions of the embodiments are simply introduced below. Apparently. The drawings in the following descriptions are merely some of the embodiments of the disclosure. Foor those skilled in the art, other drawings can be obtained according to these drawings without creative work.

FIG. 1 illustrates a flowchart of a patient information recording method according to an embodiment of the disclosure.

FIG. 2 illustrates a schematic structural diagram of a portable electronic recording device according to an embodiment of the disclosure.

FIG. 3 illustrates a schematic structural diagram of an entity recognition model according to an embodiment of the disclosure.

FIG. 4 illustrates a schematic structural diagram of a patient information recording system according to an embodiment of the disclosure.

FIG. 5 illustrates a schematic diagram of an installation position of a portable electronic recording device according to an embodiment of the disclosure.

### DETAILED DESCRIPTION OF EMBODIMENTS

The technical solutions in the disclosure are described below in conjunction with drawings.

In embodiments of the disclosure, words such as “exemplarily” and “for example” are used to indicate examples, illustrations or explanations. Any embodiment or design described as “example” in the disclosure should not be interpreted as being more preferred or more advantageous than other embodiments or designs. Specifically, the use of the word “example” is intended to present the concept in a specific way. In addition, in the embodiments of the disclosure, the meaning expressed by “and/or” can be both, or it can be either of the two.

In the embodiments of the disclosure, “image” and “picture” can sometimes be used interchangeably. It should be noted that when the difference between them is not emphasized, the meanings they intend to express are the same. “of”, “relevant” and “corresponding” can sometimes be used interchangeably. It should be noted that when the difference between them is not emphasized, the meanings they intend to express are the same.

In the embodiment of the disclosure, sometimes a subscript such as W₁ may be mistakenly written as a non-subscript form such as W1. When the difference between them is not emphasized, the meanings they intend to express are the same.

In order to make the technical problems to be solved, technical solutions and advantages of the disclosure clearer, a detailed description will be given below with reference to the drawings and specific embodiments.

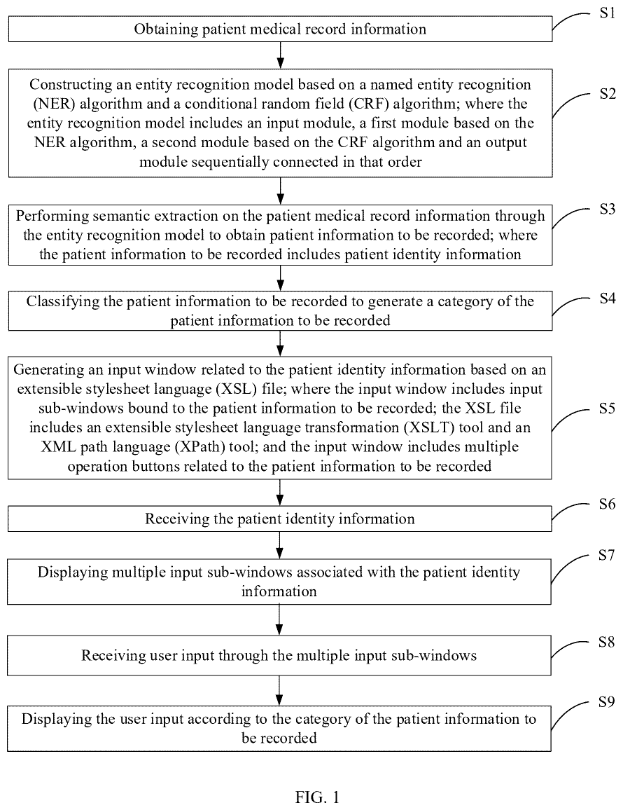

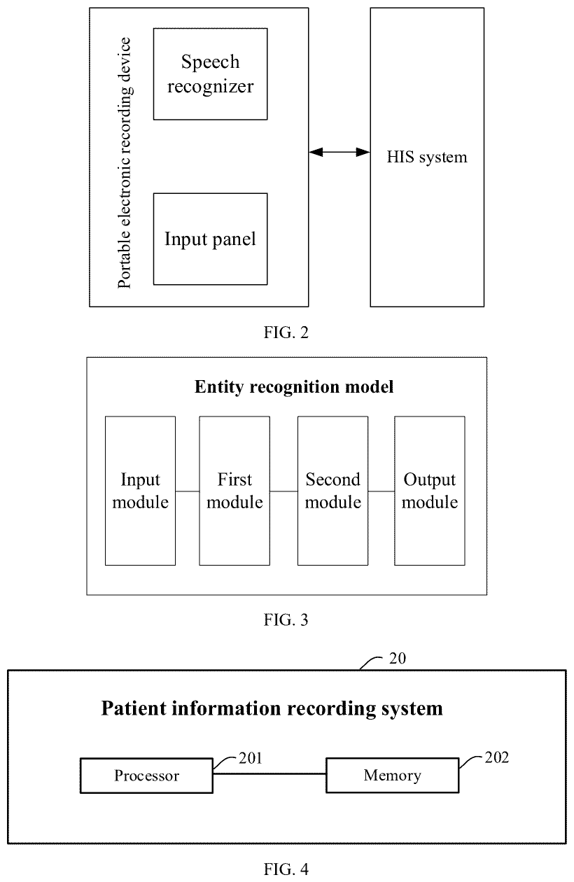

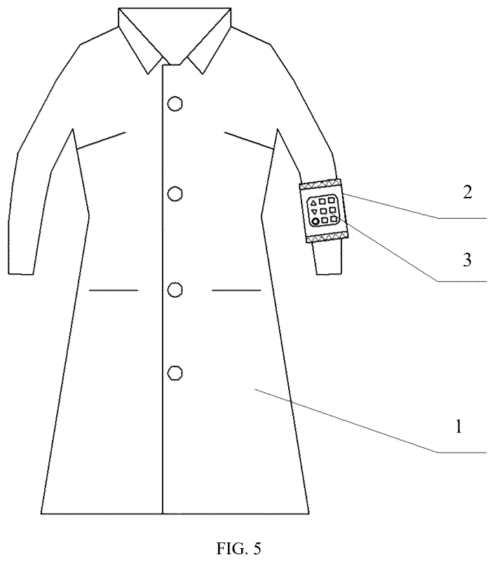

Referring to FIG. 1 of the specification, FIG. 1 illustrates a flowchart of a patient information recording method according to an embodiment of the disclosure. Referring to FIG. 2 of the specification, FIG. 2 illustrates a schematic structural diagram of a portable electronic recording device according to an embodiment of the disclosure. Referring to FIG. 3 of the specification, FIG. 3 illustrates a schematic structural diagram of an entity recognition model according to an embodiment of the disclosure.

The embodiments of the disclosure provide a patient information recording method, the method can be achieved by a patient information recording device, and the patient information recording device may be a terminal or a server. The patient information recording method is applied to a portable electronic recording device 3. The portable electronic recording device 3 is detachably disposed on a sleeve of working clothes 1 through a single-sided transparent bag 2, and the portable electronic recording device 3 is connected to a HIS system.

It should be noted that the cloth bag can be in a form of one side being transparent and the other side being cloth. Specifically, the cloth bag can be disposed on the sleeve of the work cloth in a detachable manner such as nylon adhesive buckles or zippers. The portable electronic recording device 3 is detachably disposed on the sleeve of the work cloth of medical staff through the single-sided transparent bag 2, so that the medical staff can carry it with them and record the patient information conveniently and quickly. At the same time, the portable electronic recording device 3 is connected to the HIS system to achieve real-time synchronization and data sharing of the patient information. In this way, the medical staff can record the patient information anytime and anywhere without relying on paper records or fixed electronic devices, which greatly improves convenience and efficiency of information recording. The connection between the portable electronic recording device 3 and the HIS system can enable the portable electronic recording device 3 to obtain the patient medical record information and synchronize the collected information to a shared terminal in a timely manner, so that the medical staff related to the patient can timely and uniformly check the patient medical record information and complete unified information management.

A treatment process of the patient information recording method can include the following steps S1-S9.

In step S1, patient medical record information is obtained.

Specifically, electronic medical record information or diagnosis and treatment information of a patient can be consulted through the HIS system.

In step S2, an entity recognition model is constructed based on a NER algorithm and a CRF algorithm.

Specifically, the entity recognition model includes an input module, a first module based on the NER algorithm, a second module based on the CRF algorithm and an output module sequentially connected in that order.

Specifically, the NER algorithm is a technology for recognizing named entities from text. The named entities generally refer to entities with specific meanings in the text, such as personal names, place names, and organizational names. By automatically recognizing and classifying the named entities in the text, it can help understand a semantic structure of the text, thereby achieving automated process and analysis of text information. The CRF algorithm is a statistical modeling method, which is commonly used in sequence labeling tasks, such as named entity recognition and part-of-speech tagging. Based on a conditional probability model, interdependence between input sequences is considered, and a joint probability distribution between observed sequences and labeled sequences is learned to achieve accurate modeling of sequence labeling.

Advantages of combining the NER algorithm and the CRF algorithm to construct the entity recognition model is that it comprehensively considers the context information among named entities and improves the accuracy of the entity recognition. The CRF algorithm can use the dependencies among entities in the sequence to label them in a global optimization manner, thereby avoiding a cumulative effect of local annotation errors. Secondly, the CRF algorithm can process multiple types of the named entities. The NER algorithm can usually recognize some common named entities, but may not perform well for named entities in specific fields or specific types. The CRF algorithm can better adapt to different types of named entity recognition tasks by learning features and patterns in training data. The CRF algorithm is also highly scalable. The entity recognition model based on the CRF algorithm can improve performance by introducing more features and context information, and can also adapt to data sets of different sizes and complexities. The entity recognition model based on the CRF algorithm can make full use of the context information in the text to achieve accurate recognition and labeling of the named entities, thereby providing a reliable foundation for subsequent information processing and analysis.

In step S3, semantic extraction is performed on the patient medical record information through the entity recognition model to obtain patient information to be recorded.

Specifically, the patient information to be recorded includes patient identity information.

Specifically, the patient information to be recorded refers to key information to be recorded after performing semantic extraction on the patient medical record information, which includes the patient identity information and other important information may related to medical records. The patient identity information may include basic information of the patient such as name, age, gender and identification (ID) number, which is used to identify the patient's identity. In addition, other patient information to be recorded may also involve content related to the patient medical records such as medical history, diagnosis results, treatment plan, medication, examination report of the patient. This information is an important basis for the medical staff to record and manage the patient information, and can help the medical staff better understand the condition and treatment status of the patient, so as to provide more effective medical services and management. This real-time generation can extract medical record information in different medical record formats, not just limited to a single medical record information format, and improve a scope of application of the patient information recording method.

In a possible embodiment, the step S3 specifically includes the following steps S301-S306.

In step S301, a historical patient medical record information dataset is obtained. The historical patient medical record information dataset includes multiple historical patient medical record information each with label information. The label information includes patient identity information, items to be recorded and time of the respective items to be recorded in each historical patient medical record information.

In step S302, the entity recognition model is trained by using the historical patient medical record information dataset to obtain a trained entity recognition model.

In step S303, the patient medical record information is received through the input module in the trained entity recognition model.

In step S304, specific named entities, namely the label information, in the patient medical record information are extracted through the first module in the trained entity recognition mode. The specific named entities include patient identity information, the items to be recorded and the time of the respective items to be recorded.

In a possible embodiment, the step S304 specifically includes the following steps.

A confidence parameter is introduced into the trained entity recognition model. The formulas of that specific named entities, namely the label information, in the patient medical record information are extracted through the first module in the trained entity recognition model are expressed as follows:

$$\begin{matrix}Y * & = arg {max}_{Y} P {( Y \vert X ; θ )}^{α} \\ α & = \frac{1}{1 + {e}^{- β}} \\ β & = {\sum }_{i = 1}^{N} {p}_{i}\end{matrix} ;$$

where Y* represents label information to be selected, P(Y|X; θ) represents a conditional probability of label information Y under given patient medical record information X, argmax_{Y} represents taking the label information Y maximizing the conditional probability as the label information to be selected, θ represents a model parameter, a represents an adjustable parameter, β represents the confidence parameter, namely a trust degree of the first module to the label information, e represents a base of natural logarithm, N represents a number of types of the label information, pᵢ represents a prediction probability of the first module for an i^{th} type of label information.

It should be noted that the advantage of introducing the confidence parameter is that the confidence parameter can help the entity recognition model to evaluate and select the label information more accurately, which improves reliability and accuracy of the entity recognition. By introducing the confidence parameter into the trained entity recognition model, the results can be weighted according to the confidence of the trained entity recognition model in the label information, thereby filtering out unreliable prediction results, and improving the stability of the entity recognition. Such optimized design makes the entity recognition model more reliable when processing information such as patient medical records, reduces a possibility of misidentification, thereby improving the accuracy and efficiency for recording the patient information.

The label information to be selected as the label information, namely as the specific named entities is output.

Specifically, an average prediction probability of the trained entity recognition model to the label information as a value of the confidence parameter β, which represents an overall trust degree of the first module in the trained entity recognition model for the label information. When the trained entity recognition model has high prediction probabilities for all label information, the value of the confidence parameter β will increase correspondingly, which reflects a high trust degree of the trained entity recognition model for the label information. That is, the higher the trust degree, the more accurate the selected label information, which can effectively eliminate irrelevant label information with low confidence.

In step S305, the specific named entities are input into the second module in the trained entity recognition model, and the specific named entities are corrected to obtain corrected specific named entities.

In a possible embodiment, the step S305 specifically includes the following steps.

A loss function based on the CRF algorithm is constructed, and a formula of the loss function is expressed as follows:

$$L ( θ ) = - log P ( Y \vert X ; θ ) + λ {‖ θ ‖}^{2} ;$$

where L(θ) represents the loss function under the model parameter θ, log represents a natural logarithm function, λ represents a regularization parameter, and ∥θ∥² represents taking a square norm of the model parameter.

The specific named entities are corrected through the loss function, and the corrected specific named entities corresponding to respective minimum values of the loss function are output.

It should be noted that by constructing the loss function based on the CRF algorithm, the correction of the specific named entities is achieved. The loss function can quantify the accuracy of the model prediction and adjust the model parameter in combination with the regularization parameter to minimize the value of the loss function. By minimizing the loss function, the specific named entity corresponding to the best prediction result (i.e., the minimum value of the loss function) can be found, thereby improving the performance and accuracy of the entity recognition model. This method can effectively optimize the model parameter, reduce the deviation of the prediction results, and make the entity recognition process more accurate and reliable.

In step S306, the corrected specific named entities are output as the patient information to be recorded through the output module in the trained entity recognition model.

It should be noted that multiple automated screenings to determine the patient information to be recorded can reduce the workload of the medical staff while ensuring the accuracy of the patient information to be recorded. The NER algorithm and CRF algorithm are used in combination, and the confidence parameter and the loss function optimization are introduced to improve the accuracy and reliability of the entity recognition model in extracting the patient information. During the training stage, the entity recognition model is trained by using the historical patient medical record information dataset, so that the entity recognition model can learn a wealth of medical knowledge. The introduction of the confidence parameter can help filter the unreliable prediction results and improve the stability of the entity recognition model. In the application stage, the construction of the loss function based on the CRF algorithm further optimizes the model parameter, so that the entity recognition model can more accurately identify and correct the specific named entities, thereby improving the performance and accuracy of the entity recognition. The entity recognition model can better adapt to the needs of medical information processing, and provide reliable technical support for the recording and management of the patient information.

In actual application, nursing staff need to record multiple patients during information recording, which is a heavy workload and prone to errors. In particular, information conveyed orally or in writing to the nursing staff causes serious memory workload and confusion in patient medical records, which easily leads to missing information records. Guided by the patient medical record information, the information to be recorded is automatically generated according to the time period for recording the medical record information and diagnosis and treatment information. Based on the diagnosis and treatment records of attending physicians, the nursing staff cooperates to complete the accurate patient information records. The nursing staff only needs to enter the patient's name or number to obtain the patient information to be recorded, that is, what patient data is recorded at what time, thereby optimizing and unifying the patient information management method, making the patient information recording process transparent and standardized, avoiding the problem of medical accidents that cannot be accurately attributed due to incomplete and non-standard information recorded orally or in writing, improving the management system of primary medical information, improving the efficiency and quality of medical information management links, and reducing the workload of front-line nursing staff.

In step S4, the patient information to be recorded is classified to generate a category of the patient information to be recorded.

It should be noted that the patient information to be recorded has multiple categories according to different types of patient's illness. Each type of related patient information to be recorded is classified into a category to finally obtain a clustering result, and the clustering result is a secondary classification result of the patient information to be recorded. Similar patient information to be recorded is classified as into a same larger category for easy reference and recording.

In a possible embodiment, the patient information to be recorded is classified through a K-means clustering algorithm. The step S4 specifically includes the following steps S401-S405.

In step S401, the patient information to be recorded is vectorized to obtain multiple feature vectors of the patient information to be recorded.

In step S402, a quantity of the multiple feature vectors of the patient information to be recorded and clustering centers are determined. The clustering centers are randomly selected from the multiple feature vectors of the patient information to be recorded.

In step S403, the multiple feature vectors of the patient information to be recorded are allocated to different ones of the clustering centers according to a distance nearest principle as per a formula expressed as follows:

$${c}_{i} = {argmin}_{j} {‖ {x}_{i} - {μ}_{j} ‖}^{2} ;$$

where cᵢ represents a clustering center with a nearest distance to a feature vector xᵢ of i^{th} patient information to be recorded, and μⱼ represents a j^{th} clustering center.

In step S404, an average value of feature vectors of each category of the patient information to be recorded is calculated, and clustering centers in a result of the allocated are redetermined. A formula for redetermining is as follows:

$${μ}_{j}^{′} = \frac{1}{\vert {s}_{j} \vert} {\sum }_{{x}_{i} ∈ {S}_{j}} {x}_{i} ;$$

where

$${μ}_{j}^{′}$$

represents a redetermined clustering center, Sⱼ represents a set of feature vectors where of the patient information to be recorded in the j^{th} clustering center.

In step S405, a count of iteration is increased by 1, and the steps S403-S404 are repeated until the clustering centers remain unchanged or a maximum count of iteration is reached.

The K-means clustering algorithm is a commonly used unsupervised learning algorithm, which is used to divide the dataset into K clusters, so that each data point belongs to the cluster represented by the nearest cluster center. The advantages of the K-means clustering algorithm are that it is simple and efficient, suitable for processing large-scale datasets, and the results are well interpretable, which can visually present the clustering situation of data. In addition, the K-means clustering algorithm is relatively insensitive to the selection of the initial cluster centers and can obtain stable clustering results in multiple runs, thus it has wide applicability and reliability in actual applications.

It should be noted that the advantage of using the K-means clustering algorithm to classify the patient information to be recorded is its simplicity, ease of use and high efficiency. Firstly, the K-means clustering algorithm can automatically divide the patient information to be recorded into different categories without presetting the number of categories, thereby reducing the cost of manual intervention. Secondly, the K-means clustering algorithm performs well when processing the large-scale datasets, has high computational efficiency, and can quickly classify a large amount of the patient information. In addition, the results of the K-means clustering algorithm are intuitively interpretable, and each category represents a group of patient information with similar characteristics, which helps the medical staff understand and analyze the distribution of the patient information. Therefore, the K-means clustering algorithm can improve data processing efficiency when classifying the patient information to be recorded and provide strong support for medical decision-making and management.

In step S5, an input window related to the patient identity information is generated based on an XSL file.

The input window includes input sub-windows bound to the patient information to be recorded.

The XSL file includes an XSLT tool and an XPath tool, and the input window includes multiple operation buttons related to the patient information to be recorded.

Specifically, the XSL file is a language used to describe the presentation of extensible markup language (XML) documents, which is usually used to convert the XML documents into user interfaces or other formats. The XSL file is used to generate an input window for recording the patient identity information. The XSL file is a style sheet language that can define an appearance and a layout of the input window of the patient identity information as needed. Using the XSLT tool and the XPath tool can easily customize the style and structure of the input window to meet specific needs and user preferences. The input sub-windows are bound to the patient information to be recorded to ensure that the entered data corresponds correctly to the patient identity information and avoid information confusion or entry errors. This binding relationship can improve the accuracy and reliability of data entry. The input window includes multiple operation buttons that can implement various operations on the patient information to be recorded, such as saving, editing, and deleting. These operation buttons provide more functions, so that users can easily manage and process the patient information to be recorded. The input window generated by the XSL file can be designed with a simple, intuitive, and easy-to-operate interface according to user needs. Reasonable layout and clear operation buttons enable users to quickly understand and use the input window, which improves user experience and work efficiency. Since the XSL file is based on a standard XML technology, the generated input window has good cross-platform compatibility, which means that the input window can be used across different devices and platforms, whether on desktop or mobile, the input window can maintain good display effects and functionality.

In a possible embodiment, the step S5 specifically includes the following steps S501-S510.

In step S501, the patient information to be recorded is converted to an XML through the XSLT tool.

In step S502, a page corresponding to the XML formed after the converted is used as a template to construct original components.

In step S503, label characters in the page corresponding to the XML formed after the converted are modified to custom labels, and custom components are constructed. Each custom component corresponds to a sub-input window bound to the patient information to be recorded.

In step S504, the custom components are connected to the original components.

In step S505, attributes are added to the patient information to be recorded through a form control of the XSL file. The attributes include name and category of the patient information to be recorded.

In step S506, regularization description is performed on the attributes to obtain a string set related to the attributes.

In step S507, a field corresponding to a specific named entity in the patient information to be recorded is obtained through the XPath tool.

In step S508, the field corresponding to the specific named entity is matched with the string set. Attributes corresponding to the specific named entity are maintained when the matching is successful; otherwise, attributes of the specific named entity that fails to match are constructed in the form control.

In step S509, maintained attributes and constructed attributes are transferred to the custom components.

In step S510, the custom components are submitted to obtain the input sub-windows bound to the patient information to be recorded.

It should be noted that the patient information to be recorded is converted to the XML, and the custom input windows are constructed through the XML format, which provides a convenient information entry and management tool for the medical staff. through the XML format, the information is highly structured and easy to parse and process. The creation of the custom input windows make the input information more intuitive and friendly, which is conducive to improving the work efficiency of the medical staff and the accuracy of information recording. At the same time, the use of the XSLT tool and the XPath tool can realize flexible processing and customized display of information, meet the needs of different medical scenarios, and provide reliable support for medical informatization.

In step S6, the patient identity information is received.

Specifically, the patient identity information can be the patient identity information input by the medical staff, that is, a number or name of the patient in the hospital. That is, only the patient identity information needs to be input, and the relevant contents to be recorded and the recording time point can be directly displayed, that is, with the physician's orders as the main guide, which can effectively avoid the problems of incomplete or incorrect recording of the patient information to be recorded caused by memory errors of nurse staff, and reduce the memory burden of the medical staff. In addition, it can also be effectively blamed in the division of responsibilities, that is, what is not recorded in time can be intuitively observed through the input sub-windows, which greatly improves the orderly management ability of medical processes.

In step S7, multiple input sub-windows associated with the patient identity information are displayed.

In step S8, user input is received through the multiple input sub-windows.

In a possible embodiment, the portable electronic recording device includes an input panel and a speech recognizer.

The user input includes text input and speech input.

Specifically, the user is the medical staff, that is, personnel who use the portable electronic recording device.

It can be understood that the input panel provides a function of text input, and the users can input text information through a keyboard or a touch screen. In addition, the portable electronic recording device further includes the speech recognizer, so that the users can convert oral information into text through speech input. This design can meet the needs and preferences of different users, providing a flexible and convenient way of information input, which helps to improve the work efficiency and operational convenience of the users.

In step S9, the user input is displayed according to the category of the patient information to be recorded.

In a possible embodiment, the step S9 specifically includes the following steps S901-S905.

In step S901, designated patient identity information is received.

In step S902, user input related to the designated patient identity information is obtained, and the category of patient information to be recorded of the user input is output.

In step S903, the patient information to be recorded is extracted according to the category of patient information to be recorded.

In step S904, input sub-windows bound to the patient information to be recorded are extracted to obtain extracted input sub-windows.

In step S905, the user input is displayed through the extracted sub-input windows.

It should be noted that different input sub-windows are displayed according to the categories, and the display interface has clear categories, convenient access and improved work efficiency.

Optionally, the method further includes that user input backup is generated, and the user input backup is saved to the HIS system.

It can be understood that the relevant information of the user input can be directly saved locally. In order to facilitate the internal interaction of medical information, the user input backup is saved to the HIS system to effectively ensure that different doctors of the same patient can timely access the relevant information of the patient, thereby improving information management efficiency.

The beneficial effects brought by the technical solutions provided by the embodiments of the disclosure at least include the follows.

In the disclosure, the entity recognition model is constructed based on the NER algorithm and the CRF algorithm. Specifically, the patient information to be recorded is recognized through the NER algorithm, then the recognized patient information to be recorded is checked with correct patient information to be recorded through the CRF algorithm, and the final patient information to be recorded is determined through multiple screenings, so as to reduce deviation between the patient medical record information and the recording information, and ensure the accuracy of the patient information to be recorded obtained according to the patient medical record information. This method also can be applied to electronic medical record information in different formats from different hospitals, can automatically extract the patient information to be recorded, and reduce the memory burden of the medical staff when collecting the patient information. The obtained patient information to be recorded is classified for the second time, so that the similar patient information to be recorded is classified into the same larger category, which is convenient for the medical staff to collect and access the patient information, and avoids confusion in information management. Finally, the input sub-windows bound to the patient information to be recorded are generated by using the XSL file. The input window generated by the XSL file can be easily customized according to the needs to make the interface simple and beautiful. Such input interface can make the medical staff more focused on recording the patient information, and reduce unnecessary visual interference. The patient information to be recorded is bound to the input sub-windows, and the operation buttons are provided at the same time, so that the medical staff can record the patient information intuitively and conveniently, thereby improving the work efficiency and recording quality, avoiding medical problems caused by deviations in manual memory of the medical staff, and improving information management efficiency and accuracy. In addition, the portable electronic recording device is detachably disposed on the sleeve of the work cloth through the single-sided transparent bag 2, without the need to hold a notebook or recorder. The portable electronic recording device is connected to the HIS system, which can greatly release the work efficiency of the medical staff and ensure the integrity of the recorded patient information.

Referring to FIG. 4 of the specification, FIG. 4 illustrates a schematic structural diagram of a patient information recording system according to an embodiment of the disclosure.

The disclosure further provides a patient information recording system 20, applied to the above patient information recording method. The patient information recording system includes a processor 201 and a memory 202. The memory 202 is sored with computer-readable instructions, and the computer-readable instructions are configured, when being executed by the processor 201, to implement the patient information recording method as described in the method embodiment.

The patient information recording system 20 provided by the embodiments of the disclosure can execute the above patient information recording method, and achieve the same or similar technical effects. To avoid repetition, the disclosure will not go into details.

The beneficial effects brought by the technical solutions provided by the embodiments of the disclosure at least include the follows.

In the disclosure, the entity recognition model is constructed based on the NER algorithm and the CRF algorithm. Specifically, the patient information to be recorded is recognized through the NER algorithm, then the recognized patient information to be recorded is checked with correct patient information to be recorded through the CRF algorithm, and the final patient information to be recorded is determined through multiple screenings, so as to reduce deviation between the patient medical record information and the recording information, and ensure the accuracy of the patient information to be recorded obtained according to the patient medical record information. This method also can be applied to electronic medical record information in different formats from different hospitals, can automatically extract the patient information to be recorded, and reduce the memory burden of the medical staff when collecting the patient information. The obtained patient information to be recorded is classified for the second time, so that the similar patient information to be recorded is classified into the same larger category, which is convenient for the medical staff to collect and access the patient information, and avoids confusion in information management. Finally, the input sub-windows bound to the patient information to be recorded are generated by using the XSL file. The input window generated by the XSL file can be easily customized according to the needs to make the interface simple and beautiful. Such input interface can make the medical staff more focused on recording the patient information, and reduce unnecessary visual interference. The patient information to be recorded is bound to the input sub-windows, and the operation buttons are provided at the same time, so that the medical staff can record the patient information intuitively and conveniently, thereby improving the work efficiency and recording quality, avoiding medical problems caused by deviations in manual memory of the medical staff, and improving information management efficiency and accuracy. In addition, the portable electronic recording device is detachably disposed on the sleeve of the work cloth through the single-sided transparent bag 2, without the need to hold a notebook or recorder. The portable electronic recording device is connected to the HIS system, which can greatly release the work efficiency of the medical staff and ensure the integrity of the recorded patient information.

It should be understood that the processor in the embodiments of the disclosure can be a central processing unit (CPU). The processor can also be other general-purpose processors, digital signal processors (DSP), application specific integrated circuits (ASIC), field programmable gate arrays (FPGA) or other programmable logic devices, discrete gate or transistor logic devices, or discrete hardware components. The general-purpose processor may be a microprocessor or the processor may also be any conventional processor.

It should also be understood that the memory in the embodiments of the disclosure may be a transitory memory or a non-transitory memory, or may include both transitory and non-transitory memories. Specifically, the non-transitory memory may be a read-only memory (ROM), a programmable read-only memory (PROM), an erasable programmable read-only memory (EPROM), an electrically erasable programmable read-only memory (EEPROM), or a flash memory. The transitory memory may be a random access memory (RAM), which is used as an external cache. By way of example and not limitation, many forms of the RAM are available, such as static RAM (SRAM), dynamic random access memory (DRAM), synchronous dynamic random access memory DRAM (SDRAM), double data rate synchronous dynamic random access memory (DDR SDRAM), enhanced synchronous dynamic random access memory (ESDRAM), synchronous link DRAM (SLDRAM), and direct Rambus RAM (DR RAM).

The above embodiments can be implemented in whole or in part by software, hardware (such as circuits), firmware or any other combination. When implemented by software, the above embodiments can be implemented in whole or in part in the form of a computer program product. The computer program product includes one or more computer instructions or computer programs. When the computer instructions or computer programs are loaded or executed on a computer, the process or function described in the embodiment of the disclosure is generated in whole or in part. The computer can be a general-purpose computer, a special-purpose computer, a computer network, or other programmable devices. The computer instructions can be stored in the computer-readable storage medium, or transmitted from a computer-readable storage medium to another computer-readable storage medium. For example, the computer instructions can be transmitted from a website, a computer, a server or a data center to another website, computer, server or data center by wired (such as infrared, wireless and microwave). The computer-readable storage medium can be any available medium that can be accessed by the computer or a data storage device such as a server or data center including one or more available media sets. The available medium can be a magnetic medium (for example, a floppy disk, a hard disk, and a tape), an optical medium (for example, a digital video disc abbreviated as DVD), or a semiconductor medium. The semiconductor medium can be a solid-state hard disk.

It should be understood that the term “and/or” in this article is only a description of the association relationship of associated objects, indicating that there can be three relationships. For example, A and/or B can represent that A exists alone, A and B exist at the same time, or B exists alone. A and B can be singular or plural. In addition, the character “/” in this article generally indicates that the associated objects before and after are in an “or” relationship, but it may also indicate an “and/or” relationship. Please refer to the context for specific understanding.

In the disclosure, “at least one” means one or more, and “multiple” means two or more. “At least one of the following” or similar expressions refers to any combination of these items, including any combination of single or plural items. For example, at least one of a, b, and c can be represented by a, b, c, a-b, a-c, b-c, or a-b-c. Where a, b, and c can be single or multiple.

It should be understood that in various embodiments of the disclosure, the size of the serial numbers of the aforementioned processes does not mean the execution order. The execution order of each process should be determined by its function and internal logic, and should not constitute any limitation on the implementation process of the embodiments of the disclosure.

Those skilled in the art will appreciate that the units and algorithm steps of each example described in conjunction with the embodiments disclosed herein can be implemented in electronic hardware, or a combination of computer software and the electronic hardware. Whether these functions are performed in hardware or software depends on the specific application and design constraints of the technical solutions. Those skilled in the art can use different methods to implement the described functions for each specific application, but such implementation should not be considered to be beyond the scope of the disclosure.

Those skilled in the art can clearly understand that, for the convenience and brevity of description, the specific working processes of the above-described apparatuses, devices and units can refer to the corresponding processes in the aforementioned method embodiments and will not be repeated here.

In the embodiments provided by the disclosure, it should be understood that the disclosed devices, apparatuses and methods can be implemented in other ways. For example, the device embodiments described above are only exemplary. For example, the division of the units is only a logical function division. There may be other division methods in actual implementation, such as multiple units or components can be combined or integrated into another device, or some features can be ignored or not executed. Another point is that the mutual coupling or direct coupling or communication connection shown or discussed can be through some interfaces, indirect coupling or communication connection of devices or units, which can be electrical, mechanical or other forms.

The units described as separate components may or may not be physically separated, and the components shown as units may or may not be physical units, that is, they may be located in one place or distributed on multiple network units. Some or all of the units may be selected according to actual needs to achieve the purpose of the solution of this embodiment.

In addition, each functional unit in each embodiment of the disclosure may be integrated into one processing unit, or each unit may exist physically separately, or two or more units may be integrated into one unit.

If the functions are implemented in the form of software functional units and sold or used as independent products, they can be stored in a computer-readable storage medium. Based on this understanding, the technical solutions of the disclosure can be embodied in the form of a software product in essence, in the part that contributes to the related art, or in the part of the technical solutions. The computer software product is stored in a storage medium, including multiple instructions for enabling a computer device (which can be a personal computer, a server, or a network device) to execute all or part of the steps of the methods described in each embodiment of the disclosure. The aforementioned storage medium includes U-disk, mobile hard disk, ROM, RAM, disk or optical disk, and other media that can store program codes.

The embodiments of the disclosure provide a non-transitory computer-readable medium stored with a computer program, the computer program is configured, when being executed by a processor, to implement the patient information recording method as described in the method embodiment.

The non-transitory computer-readable medium provided by the disclosure can implement the steps and effects of the patient information recording method as described in the method embodiment. To avoid repetition, the disclosure will not go into details.

The beneficial effects brought by the technical solutions provided by the embodiments of the disclosure at least include the follows.

In the disclosure, the entity recognition model is constructed based on the NER algorithm and the CRF algorithm. Specifically, the patient information to be recorded is recognized through the NER algorithm, then the recognized patient information to be recorded is checked with correct patient information to be recorded through the CRF algorithm, and the final patient information to be recorded is determined through multiple screenings, so as to reduce deviation between the patient medical record information and the recording information, and ensure the accuracy of the patient information to be recorded obtained according to the patient medical record information. This method also can be applied to electronic medical record information in different formats from different hospitals, can automatically extract the patient information to be recorded, and reduce the memory burden of the medical staff when collecting the patient information. The obtained patient information to be recorded is classified for the second time, so that the similar patient information to be recorded is classified into the same larger category, which is convenient for the medical staff to collect and access the patient information, and avoids confusion in information management. Finally, the input sub-windows bound to the patient information to be recorded are generated by using the XSL file. The input window generated by the XSL file can be easily customized according to the needs to make the interface simple and beautiful. Such input interface can make the medical staff more focused on recording the patient information, and reduce unnecessary visual interference. The patient information to be recorded is bound to the input sub-windows, and the operation buttons are provided at the same time, so that the medical staff can record the patient information intuitively and conveniently, thereby improving the work efficiency and recording quality, avoiding medical problems caused by deviations in manual memory of the medical staff, and improving information management efficiency and accuracy. In addition, the portable electronic recording device is detachably disposed on the sleeve of the work cloth through the single-sided transparent bag 2, without the need to hold a notebook or recorder. The portable electronic recording device is connected to the HIS system, which can greatly release the work efficiency of the medical staff and ensure the integrity of the recorded patient information.

The aforementioned is only specific embodiments of the disclosure, but the protection scope of the disclosure is not limited thereto. Any those skilled in the art can easily think of changes or substitutions within the technical scope disclosed by the disclosure, which should be included in the protection scope of the disclosure. Therefore, the protection scope of the disclosure should be based on the protection scope of the claims.

There are a few points to note as follows.
(1) The drawings of the embodiments of the disclosure only relate to the structures related to the embodiments of the disclosure, and other structures may refer to the general design.
(2) For the sake of clarity, in the drawings used to describe the embodiments of the disclosure, the thickness of the layers or regions is exaggerated or reduced, that is, these drawings are not drawn according to the actual scale. It can be understood that when an element such as a layer, film, region or substrate is referred to as being located “on” or “under” another element, the element may be located “directly” “on” or “under” another element or there may be an intermediate element.
(3) In the absence of conflict, the embodiments of the disclosure and the features therein may be combined with each other to obtain new embodiments.

The above are only specific embodiments of the disclosure, but the protection scope of the disclosure is not limited thereto. The protection scope of the disclosure shall be based on the protection scope of the claims.

1. A patient information recording method, applied to a portable electronic recording device, wherein the portable electronic recording device is detachably disposed on a sleeve of working clothes through a single-sided transparent bag and connected to a hospital information system (HIS), and the patient information recording method comprises:
S1, obtaining patient medical record information;
S2, constructing an entity recognition model based on a named entity recognition algorithm and a conditional random field algorithm, wherein the entity recognition model comprises an input module, a first module based on the named entity recognition algorithm, a second module based on the conditional random field algorithm and an output module sequentially connected in that order;
S3, performing semantic extraction on the patient medical record information through the entity recognition model to obtain patient information to be recorded, wherein the patient information to be recorded comprises patient identity information;
S4, classifying the patient information to be recorded to generate a category of the patient information to be recorded;
S5, generating an input window related to the patient identity information based on an extensible stylesheet language (XSL) file, wherein the input window comprises input sub-windows bound to the patient information to be recorded, the XSL file comprises an extensible stylesheet language transformation (XSLT) tool and an XML path language (XPath) tool, and the input window comprises a plurality of operation buttons related to the patient information to be recorded;
S6, receiving the patient identity information;
S7, displaying a plurality of input sub-windows associated with the patient identity information;
S8, receiving user input through the plurality of sub-input windows; and
S9, displaying the user input according to the category of the patient information to be recorded;
wherein the S3 specifically comprises:
S301, obtaining a historical patient medical record information dataset; wherein the historical patient medical record information dataset comprises a plurality of historical patient medical record information each with label information, and the label information comprises patient identity information, items to be recorded and time of the respective items to be recorded in each of the plurality of historical patient medical record information;
S302, training the entity recognition model by using the historical patient medical record information dataset to obtain a trained entity recognition model;
S303, receiving the patient medical record information through the input module in the trained entity recognition model;
S304, extracting specific named entities, namely the label information, in the patient medical record information through the first module in the trained entity recognition model, wherein the specific named entities comprise the patient identity information, the items to be recorded and the time of the respective items to be recorded;
S305, inputting the specific named entities into the second module in the trained entity recognition model, and correcting the specific named entities to obtain corrected specific named entities; and
S306, outputting the corrected specific named entities as the patient information to be recorded through the output module in the trained entity recognition model;
wherein the S304 specifically comprises:
introducing a confidence parameter into the trained entity recognition model, and extracting the specific named entities in the patient medical record information through the first module in the trained entity recognition model as per formulas expressed as follows:
$$\begin{matrix}Y * & = arg {max}_{Y} P {( Y \vert X ; θ )}^{α} \\ α & = \frac{1}{1 + {e}^{- β}} \\ β & = {\sum }_{i = 1}^{N} {p}_{i}\end{matrix} ;$$
where, Y* represents label information to be selected, P(Y|X; θ) represents a conditional probability of label information Y under given patient medical record information X, argmax_{Y} represents taking the label information Y maximizing the conditional probability as the label information to be selected, θ represents a model parameter, a represents an adjustable parameter, β represents the confidence parameter, namely a trust degree of the first module to the label information, e represents a base of natural logarithm, N represents a number of types of the label information, pᵢ represents a prediction probability of the first module for an i^{th} type of label information; and
outputting the label information to be selected as the label information, namely as the specific named entities;
wherein the S305 specifically comprises:
constructing, based on the conditional random field algorithm, a loss function expressed as follows:
$$L ( θ ) = - log P ( Y \vert X ; θ ) + λ {‖ θ ‖}^{2} ;$$
where, L(θ) represents the loss function under the model parameter θ, log represents a natural logarithm function, λ represents a regularization parameter, and ∥θ∥² represents taking a square norm of the model parameter θ; and
correcting the specific named entities through the loss function, and outputting the corrected specific named entities corresponding to respective minimum values of the loss function;
wherein the patient information to be recorded is classified through a K-means clustering algorithm, and the S4 specifically comprises:
S401, vectorizing the patient information to be recorded to obtain a plurality of feature vectors of the patient information to be recorded;
S402, determining a quantity of the plurality of feature vectors of the patient information to be recorded and clustering centers, wherein the clustering centers are randomly selected from the plurality of feature vectors of the patient information to be recorded;
S403, allocating the plurality of feature vectors of the patient information to be recorded to different ones of the clustering centers according to a distance nearest principle as per a formula expressed as follows:
$${c}_{i} = {argmin}_{j} {‖ {x}_{i} - {μ}_{j} ‖}^{2} ;$$
where, cᵢ represents a clustering center with a nearest distance to a feature vector xᵢ of i^{th} patient information to be recorded, and μⱼ represents a j^{th} clustering center;
S404, calculating an average value of feature vectors of each category of the patient information to be recorded, and redetermining clustering centers in a result of the allocating as per a formula expressed as follows:
$${μ}_{j}^{′} = \frac{1}{\vert {s}_{j} \vert} {\sum }_{{x}_{i} ∈ {S}_{j}} {x}_{i} ;$$
where, μⱼ represents a redetermined j^{th} clustering center, Sⱼ represents a set of feature vectors of the patient information to be recorded in the j^{th} clustering center; and
S405, increasing a count of iteration by 1, and repeating the S403 and the S404 until the clustering centers remain unchanged or a maximum count of iteration is reached; and
wherein the S5 specifically comprises:
S501, converting the patient information to be recorded to an extensible markup language (XML) through the XSLT tool;
S502, constructing original components by using a page corresponding to the XML formed after the converting as a template;
S503, modifying label characters, in the page corresponding to the XML formed after the converting, to custom labels, and constructing custom components; wherein each of the custom components corresponds to one of the input sub-windows bound to the patient information to be recorded;
S504, connecting the custom components to the original components;
S505, adding attributes to the patient information to be recorded through a form control of the XSL file, wherein the attributes comprise name and category of the patient information to be recorded;
S506, performing regularization description on the attributes to obtain a string set related to the attributes;
S507, obtaining a field corresponding to a specific named entity in the patient information to be recorded through the XPath tool;
S508, matching the field corresponding to the specific named entity with the string set, maintaining attributes corresponding to the specific named entity when the matching is successful, otherwise, constructing attributes corresponding to the specific named entity that fails to match in the form control;
S509, transferring maintained attributes and constructed attributes to the custom components; and
S510, submitting the custom components to obtain the input sub-windows bound to the patient information to be recorded.

2-6. (canceled)

7. The patient information recording method as claimed in claim 1, wherein the portable electronic recording device comprises an input panel and a speech recognizer; and the user input comprises text input and speech input.

8. The patient information recording method as claimed in claim 1, wherein the S9 specifically comprises:
S901, receiving designated patient identity information;
S902, obtaining user input related to the designated patient identity information, and outputting the category of patient information to be recorded of the user input;
S903, extracting the patient information to be recorded according to the category of the patient information to be recorded;
S904, extracting input sub-windows bound to the patient information to be recorded to obtain extracted input sub-windows; and
S905, displaying the user input through the extracted input sub-windows.

9. A patient information recording system, comprising:
a processor; and
a memory stored with computer-readable instructions, wherein the computer-readable instructions are configured, when being executed by the processor, to implement the patient information recording method as claimed in claim 1.

10. A non-transitory computer-readable medium stored with a computer program, wherein the computer program is configured, when being executed by a processor, to implement the patient information recording method as claimed in claim 1.
